(19) United States
(12) Patent Application Publication
(10) Pub. No.: US 2013/0053338 A1
(43) Pub. Date: Feb. 28, 2013

(54) Use of P2X Purinergic Receptor Agonists to Enhance Insulin Secretion in Pancreatic Beta Cells

(71) Applicants: Per-Olof Berggren, Solna (SE); Alejandro Caicedo, Coral Gables, FL (US); Caroline M. Jacques-Silva, Miami Beach, FL (US)

(72) Inventors: Per-Olof Berggren, Solna (SE); Alejandro Caicedo, Coral Gables, FL (US); Caroline M. Jacques-Silva, Miami Beach, FL (US)

(21) Appl. No.: 13/580,232

(22) PCT Filed: Mar. 21, 2011
(86) PCT No.: PCT/US2011/029260

§ 371 Date: Oct. 12, 2012

Related U.S. Application Data
(60) Provisional application No. 61/315,612, filed on Mar. 19, 2010.

(51) Int. Cl. A61K 31/7076; G01N 33/566; C07H 19/10; A61P 5/50; A61K 31/7072; C07H 19/20

(52) U.S. Cl. 514/47; 514/51; 536/26.7; 536/26.8; 435/7.21

(57) ABSTRACT

Pharmaceutical compositions containing P2X purinergic agonists, e.g. P2X3 agonists, for increasing insulin secretion in a subject, methods of use, and methods of screening for related compounds and agents.

The invention described herein was made with U.S. government support under Grant No. 1RO3DK075487, awarded by the National Institutes of Health/NIDDK. The U.S. government has certain rights in the invention,

### INTRODUCTION

Diabetes mellitus is a widespread metabolic disorder characterized by high blood sugar and defects in insulin regulation. Although a number of treatments are available, the condition remains poorly controlled in many patients. Thus, there is a need for new treatments and new effective pharmaceutical compounds for use as primary or adjuvant therapeutics.

Glucose homeostasis is tightly controlled by hormone secretion from the endocrine part of the pancreas, the islets of Langerhans. Even small physiological deviations (e.g. 10%) in plasma glucose are effectively counteracted by sharp (e.g. threefold) increases in the secretion of the islet hormones insulin or glucagon (1). Intra-islet autocrine and paracrine signaling are pivotal mechanisms for proper function of the islet, making islet cells extremely sensitive and responsive to plasma glucose fluctuations. The roles of different compounds such as GABA, glutamate, Zn²⁺, insulin, and ATP as autocrine and paracrine regulators of islet hormone release have been examined extensively (2-8). Among the different factors thought to regulate hormone release, extracellular ATP seems important because it is present in insulin-containing granules and it is released during glucose stimulation in sufficient amounts to stimulate ATP receptors. Extracellular ATP is an important neurotransmitter signal in the brain, as well as in vascular, endocrine and immune cells (13-15). The purinergic system comprises receptors for extracellular ATP and adenosine, the P2 and P1 receptors, respectively. P2 purinergic receptors can be divided into two categories, i.e. the metabotropic P2Y receptors (G-protein coupled) and the ionotropic P2X receptors (ligand-gated ion channels) (16). The ionotropic P2X family comprises seven subtypes designated P2X₁-P2X₇ that regulate cell function by opening cation channels permeable to Na⁺, K⁺, and Ca²⁺ (15, 17). Activation of these channels regulates the release of neurotransmitters and hormones, either through direct Ca²⁺ influx or by promoting membrane depolarization and thereby, induction of action potentials (18-21).

The role of purinergic signaling in the physiology of pancreatic islets has been studied in rodent models, but the results in the literature are conflicting (22-28). In rat islets, purinergic agonists have been reported to increase insulin secretion (22, 28). This contrasts with a report on rat islets showing that extracellular ATP provides excitatory as well as inhibitory feedback loops for insulin secretion (23). In mouse islets, extracellular ATP has been consistently reported to decrease glucose-induced insulin secretion (24-26). In the two reports on human islets, purinergic agonists were shown to evoke inward currents in β cells and to stimulate insulin release (29, 30), but the receptors involved were not identified. More importantly, the physiological contexts under which these receptors are activated have not been investigated.

In rodent islets, insulin granules contain ATP, and ATP is coreleased with insulin during high glucose stimulation, reaching extracellular concentrations >25 μM (9-12, 33). Recent papers have provided evidence that smaller molecules such as ATP can he released by a kiss-and-run exocytotic mechanism, whereas insulin is retained in the granule (12, 34). Furthermore, insulin secretion shows a lower activation threshold in human islets than in mouse islets, and slight increases in insulin secretion already occur at 3 mM glucose (FIG. 6; see also ref 35). Thus, ATP is likely to be coreleased with insulin at relatively low glucose concentrations. ATP is, therefore, an excellent signaling candidate for modulating the β-cell responsiveness to increases in glucose around the threshold.

### SUMMARY

Because islets from different species are strikingly different in terms of structure and function and because the data on purinergic signaling in islet biology are not conclusive, we decided to study in detail the role of purinergic signaling in human beta cells. We were particularly interested in defining the role of endogenously released ATP during stimulation of beta cells with increases in glucose concentration. We examined the effect of ATP signaling by performing dynamic hormone release assays, imaging of cytoplasmic free Ca²⁺ concentration ([Ca²⁺]ᵢ), RT-PCR, and immunohistochemistry. Our results demonstrate that human beta cells express P2X receptors that induce Ca²⁺ influx and insulin secretion, promoting autocrine positive feedback during glucose-induced insulin release.

P2X receptors in beta cells are therefore rational targets for drugs to enhance insulin secretion. Contrary to other therapies, activation of P2X receptors likely enhances endogenous insulin secretion when beta cell are activated, that is, in the appropriate physiological context. We expect that modulation of P2X receptors in beta cells will be an adjuvant therapy in the management of drug-treated diabetes.

Modulation of P2X receptor activity has emerged as a potential point of therapeutic intervention in diseases such as lower urinary tract dysfunction and irritable bowel syndrome. The information derived from our studies indicates that P2X receptors are also rational targets for drugs that could be used to improve glycemic control alone or in combination with oral hypoglycemic agents (e.g. sulphonylureas) or with basal insulin supplement in the context of type 2 diabetes. We expect this therapy to reduce diabetic morbidity in people with type 2 diabetes.

By using positive modulators of P2X receptors, we intervene with a natural mechanism amplifying insulin secretion, which is compromised in diabetes. In contrast to current approaches, our therapy enhances endogenous insulin secretion in the appropriate physiological context.

Accordingly, the invention provides a method of increasing insulin secretion in a subject in need thereof, by administering an effective amount of a P2X purinergic agonist (e.g. 2-methylthio-ATP (2-meSATP), 5-bromouridine 5-triphosphate, a benzoyl-benzoyl ATP, such as 3′-O-(4-benzoylbenzoyl)-ATP, α,β-methylene ATP, 2-meSATP, α,β-methylene ATP, or BzATP(2′(3′)-O-(4-Benzoylbenzoyl)ATP)). BzATP may be considered the least toxic of these purinergic agonists.

The subject may be any mammal that is subject to conditions in which increased insulin secretion may be desirable, particularly a primate, e.g. a human. In one embodiment, the subject is suffering from diabetes mellitus, e.g. type 2 diabetes. In one preferred embodiment, the P2X purinergic agonist is a P2X₃ agonist, for example 2-methylthio-ATP (2-meSATP), 5-bromouridine 5-triphosphate, 3′-O-(4-benzoyIbenzoyl)-ATP, and α,β-methylene ATP.

Appropriate dosages of P2X purinergic agonist can be determined by routine experimentation by those of skill in the art. In one embodiment, dosages are expected to result in a concentration at the target tissue of between about 10 μM and 1 mM, e.g. between about 10 μM and 100 μM.

Also provided is a use of a P2X purinergic agonist in a pharmaceutical composition for increasing insulin secretion in a subject in need thereof, for example a subject, e.g. a human, suffering from diabetes mellitus, e.g. type 2 diabetes. In one embodiment the P2X purinergic agonist is a P2X₃ agonist, e.g. selected from the group consisting of 2-methylthio-ATP (2-meSATP), 5-bromouridine 5-triphosphate, 3′-O-(4-benzoylbenzoyl)-ATP, and α,β-methylene ATP.

Also provided is a pharmaceutical composition comprising an effective amount of a P2X purinergic agonist, e.g. a P2X₃ agonist, to stimulate insulin secretion for treatment of diabetes. The P2X₃ agonist may be selected, for example, from the group consisting of 2-methylthio-ATP (2-meSATP), 5-bromouridine 5-triphosphate, 3′-O-(4-benzoylbenzoyl)-ATP, and α,β-methylene ATP.

The pharmaceutical compositions to be administered in accordance with the invention optionally include pharmaceutically acceptable diluents, carriers and excipients as is customary in the pharmaceutical arts.

The invention also provides a means of screening for drugs/compounds to be used in the methods of the invention, by screening test compounds for their ability to act specifically on the P2X3 receptor in the beta cell. Compounds can be screened for activity as P2X3 agonists according to the methods described herein, and compounds that exhibit such activity can be selected for further testing in vitro and in vivo to determine whether they are good candidates for pharmaceutical agents to increase insulin secretion. Therefore, also provided is a screening method for detecting a compound/agent with efficacy in increasing insulin secretion in a mammal, particularly a primate, e.g. a human, comprising contacting the compound with a P2X3 receptor and measuring the activity of the receptor, e.g. by measuring an increase/decrease in insulin secretion of a cell bearing the receptor. Compounds/agents that stimulate P2X3receptor activity will be considered as potential compounds for increasing insulin secretion and for inclusion in pharmaceutical compounds.

### Definitions

As used herein, “about” is intended to mean +/−10%.

By “pharmaceutically acceptable diluents, excipients and carriers” is meant such compounds as will be known to persons of skill in the art as being compatible with the pharmaceutical compositions and suitable for local or systemic administration to an animal, particularly a human or other primate, according to the invention.

As used herein, the terms “treatment,” “treating,” etc., refer to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or any adverse affect attributable to the condition or disease. “Treatment,” thus, for example, covers: (a) preventing the condition or disease from occurring in an individual who is predisposed to the condition or disease but has not yet been diagnosed as having it; (b) inhibiting the condition or disease, such as, arresting its development; and (c) relieving, alleviating or ameliorating the condition or disease, such as, for example, causing regression of the condition or disease in an individual who is afflicted with the condition or disease, e.g. has been diagnosed by a Medical practitioner.

By “target tissue” is meant a tissue or cell group wherein the compounds of the invention exert a therapeutic effect, e.g. pancreas, or pancreas islet cell.

The term “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and is compatible with other ingredients of the formulation. For example, the carrier for a formulation containing the present therapeutic compounds and compositions preferably does not include oxidizing agents and other compounds that are known to be deleterious to such. Suitable carriers include, but are not limited to, water, dextrose, glycerol, saline, ethanol, buffer, dimethyl sulfoxide, Cremaphor EL, and combinations thereof. The carrier may contain additional agents such as wetting or emulsifying agents, or pH buffering agents. Other materials such as anti-oxidants, humectants, viscosity stabilizers, and similar agents may be added as necessary.

Pharmaceutically acceptable salts herein include the acid addition salts (e.g. formed with a free amino group) and which are formed with inorganic acids, including, but not limited to hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, and histidine.

The term “pharmaceutically acceptable excipient,” includes vehicles, adjuvants, or diluents or other auxiliary substances, such as those conventional in the art, which are readily available to the public. For example, pharmaceutically acceptable auxiliary substances include pH adjusting and buffering agents, tonicity adjusting agents, stabilizers, wetting agents and the like.

As used herein, the singular forms “a”, “an”, and “the” include plural forms unless the context clearly dictates otherwise. Thus, for example, reference to “a compound” includes a plurality of such compounds.

As mentioned above, effective amounts of the pharmaceutical compounds are administered to an individual, where “effective amount” means a dosage sufficient to produce a desired result. In some embodiments, the desired result is stimulation of insulin secretion to a desirable level. The amount of the therapeutic agent to be administered varies depending upon the manner of administration, the age and body weight of the subject/patient, and with the subject's symptoms and condition. A compound is administered at a dosage that best achieves medical goals with the fewest corresponding side effects.

Typically, the compositions to be used in the instant invention will contain from less than about 1% up to about 99% of the active ingredient(s). The appropriate dose to be administered depends on the subject to be treated, such as the general health of the subject, the age of the subject, the state of the disease or condition, the weight of the subject, etc.

The pharmaceutically acceptable excipients, such as vehicles, adjuvants, carriers or diluents, are conventional in the art. Suitable excipient vehicles are, for example, water, saline, dextrose, glycerol, ethanol, or the like, and combinations thereof. In addition, if desired, the vehicle may contain minor amounts of auxiliary substances such as pH adjusting and buffering agents, tonicity adjusting agents, stabilizers, wetting agents or emulsifying agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the individual being treated.

The therapeutic compounds can be formulated into preparations for injection by dissolving, suspending or emulsifying them in an aqueous or non-aqueous solvent, such as vegetable or other similar oils, including corn oil, castor oil, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Conventional routes of administration will be evident to the skilled worker. These include, e.g., oral or subcutaneous administration. Other routes of administration include rectal, transdermal, intravenous, intramuscular, respiratory (e.g. through an inhalation device) intranasal, and the like.

Effective dosages can be determined by routine, conventional procedures. As examples, BzATP or α,β-methylene ATP can be administered at a concentration of about 50 μM.

Patents and other publications cited herein are hereby incorporated by reference.

This application claims the priority of U.S. provisional application No. 61/315,612, filed Mar. 19, 2010, which is hereby incorporated by reference.

### BRIEF DESCRIPTION OF THE DRAWINGS

FIG. 1. ATP is secreted by human islets at low glucose concentrations, and it amplifies insulin secretion during glucose stimulation. (A) The ectonucleotidase inhibitor ARL67156 (50 μM) increased insulin secretion at a low glucose concentration (3 mM; green symbols). Apyrase (5 U/mL) did not change basal insulin secretion (red symbols). Average traces of insulin secretion are shown (n=4 perifusions). Control, black symbols. Bar indicates drug application. Data in all figures are presented as average±SEM. (B) Quantification of the results shown in A. Δ[Insulin] (μU/μg DNA), change in insulin secretion from prestimulus levels. (C) Insulin secretion induced by raising glucose from 3 mM to 11 mM (black symbols) was reduced in the presence of apyrase (5 U/mL; red symbols). Average traces of insulin secretion are shown (n=4 perifusions). 11G indicates 10 min of elevated glucose (11 mM). (D) Quantification of the results shown in C. Reducing extracellular ATP levels with apyrase (5 U/mL) decreased glucose-induced insulin release by ˜15%. Adding adenosine deaminase (ADA; 1 U/mL) to degrade adenosine did not change the effect of apyrase on glucose-stimulated insulin secretion. Control is insulin secretion induced by elevating glucose from 3 mM to 11 mM. Asterisks denote statistical significance (ANOVA followed by multiple comparisons versus control group in Bonferroni t test; P<0.05).

FIG. 2. Endogenously released ATP amplifies glucose-induced insulin secretion in human islets through P2X receptors. (A) Insulin secretion induced by raising glucose from 3 mM to 11 mM was reduced in the presence of the P2X receptor antagonists iso-PPADS (50 μM; red symbols) and oATP (500 μM; green symbols; representative traces of at least three perifusions). Bar denotes antagonist application. 11G indicates 10 min of elevated glucose (11 mM). (B) Quantification of the results shows the effects of suramin (100 μM), iso-PPADS (50 μM); oATP (500 μM), MRS2159 (10 μM), Brilliant Blue G (BBG; 1 μM), KN-62 (1 μM), reactive blue 2 (RB2; 50 μM), and MRS2179 (10 μM) on the magnitude of glucose-induced insulin response (peak amplitudes; n 3). Suramin, iso-PPADS, and oATP reduced insulin release by 40%, 30%, and 65%, respectively. The specificity of the antagonists is indicated at the top of the panel. Asterisks denote statistical significance (ANOVA followed by multiple comparisons versus control group in Bonferroni t test; P<0.05). (C) ATP concentration-response relationships for insulin secretion in human (n=3 islet preparations; black and blue symbols are 3 mM and 11 mM glucose, respectively) and rat islets (n=3; red symbols) are shown. Control is nonstimulated basal insulin secretion. (D) The purinergic agonists ATP (100 μM), ATPγS (50 μM), BzATP (50 μM), and ADP (100 μM) elicited insulin secretory responses in human islets at low glucose concentrations (3 mM). The P2Y agonist UTP (100 μM) and the P1 receptor agonist adenosine (Ado; 100 M) did not evoke strong insulin responses (n≧3 islet preparations).

FIG. 3. P2X Expression profiles in human islets. (A) In situ hybridization on human pancreatic sections with riboprobes for all P2X receptors showed expression of P2X3, P2X5, and P2X7 mRNA in islets (Upper). No hybridization signal could be detected for P2X1, P2X2, P2X4, or P2X6. The hybridization signal for P2X3 colocalized with insulin immunoreactivity (Lower). (Scale bar, 50 μm.) Images are representative of three human pancreata. (B) Confocal images of human pancreatic sections showing immunoreactivity for P2X3 in islets. P2X3 immunoreactivity (green) localized to insulin-expressing β cells [red; Right, higher magnification image of region indicated by Left) is shown. Cell nuclei are shown in gray. Images are representative of five human pancreata. (Scale bar, 20 μm.) (C) Western blotting analysis of lysates from human (HI) and monkey islets (MI) with human (HB) and monkey brain (MB) used as positive controls. A band for P2X3 receptors is visible at ˜65 kDa (Upper). Specific bands disappeared when primary antibodies were pre-adsorbed with their cognate protein (Lower). Arrows indicate 50 kDa molecular weight (n=3 islet preparations). A molecular marker was run in parallel. (D) ATP S (50 μM) induced [Ca²⁺]ᵢ responses in individual human islet cells loaded with Fura-2. These cells responded to stimulation, with high glucose (11 mM; black traces, representative of 8 cells). Most of the alpha cells, identified by their response to kainate (100 μM), did not respond to ATP S (gray traces; representative of 25 cells). Bars indicate the duration of the stimulus. The graph (Right) shows the percentages of cells that responded to ATP S in the glucose-responsive (11G; n=8) and kainate-responsive cell populations (Kai; n=25). Recorded at low glucose concentration (3 mM).

FIG. 4. ATP-induced insulin release by human β cells requires P2X receptor activation and Ca²⁺ influx through voltage-gated Ca²⁺ channels. (A) Insulin secretion induced by ATP (10 μM) was inhibited in the presence of iso-PPADS (50 μM). Average traces from three islet preparations ±SEM with (red symbols) and without (black symbols) incubation in iso-PPADS. Bars indicate drug or antagonist application. (B) Insulin secretion included by ATP (10 μM) was reduced in nominal 0 Ca²⁺ (+1 mM EGTA; red symbols) or in the presence of the Ca²⁺ channel blockers Cd²⁺ (100 μM; blue symbols) or nifedipine (Nife; 10 μM; gray symbols). Thapsigargin treatment (Thapsi; 1 μM; green symbols) did not affect insulin responses. Average insulin response of three islet preparations (±SEM) before (Left) and during treatment (Right). Con, control insulin response to ATP (black symbols), (C) Iso-PPADS reduced [Ca²⁺]i responses induced by ATPγS (50 μM) in human β cells. Only islet cells that responded to high glucose (16 mM) were examined. Bars indicate the duration of the stimulus or antagonist application. Average trace was 7 cells±SEM. (P) [Ca²⁺]i responses induced by ATPγS (50 μM) were reduced in nominal 0 Ca²⁺ (+1 mM EGTA) or in the presence of nifedipine (10 μM). [Ca²⁺]i responses to ATPγS were not decreased in the presence of thapsigargin (1 μM). There was an average peak response amplitude±SEM of 3 cells from three human islet preparations. Asterisks denote statistical significance (Student t test; P<0.05). Con, control [Ca²⁺]i response to ATPγS before treatment; AUC, area under the curve.

FIG. 5. Proposed model for the positive autocrine feedback loopmediated by ATP in human β cells. ATP, coreleased with insulin, activates ionotropic P2X3 receptors in the β-cell plasma membrane. This opens the cation selective P2X3 channel pore to let Na⁺ and Ca²⁺ flow into the cell (1). The resultant membrane depolarization and increase in action potential frequency increases Ca²⁺ flux through high voltage-gated Ca2+ channels. Increased [Ca²⁺]i (2) stimulates insulin secretion. In the absence of P2X3 activation, insulin secretion is diminished (Right).

FIG. 6. Slight increases in insulin secretion occurring at low glucose concentrations. Insulin secretion was stimulated in human islets by raising the glucose concentration from 1 mM to 3 mM. Average traces of insulin secretion are shown (n=8 perifusions).

FIG. 7. Species differences in ATP-induced insulin secretion. Monkey islets (black symbols) responded to increasing concentrations of ATP like human islets. No responses to ATP were observed in mouse (red symbols), rat (blue symbols), or pig (green symbols) islets in the range of concentrations tested (1-1,000 μM). Shown are representative experiments (n≧3 islet preparations per species).

FIG. 8. ATP elicits small increases in glucagon secretion, (Left) Glucagon responses to ATP were small in monkey islets (blue symbols) and human islets (black symbols) and were difficult to discern in mouse (red symbols) or pig islets (green symbols). Shown are representative experiments from greater than or equal to two islet preparations per species. (Right) P2X3 immunoreactivity, shown on the left, was not present in alpha cells (glucagon immunostaining; red). Confocal images of human pancreatic sections showing immunoreactivity for P2X4 (green) in alpha cells are on the right. (Scale bar, 20 μm)

### DETAILED DESCRIPTION

### Experimental Procedures

Islet Isolation. Islets were isolated as previously described (57). Monkey islets were isolated from cynomolgus monkeys (Macacca fascicularis)>4 years of age at the time of pancreas procurement, as previously described (58). Pig pancreata were procured from the local slaughterhouse. Mice (C57BL/6) and rat (Lewis rat; Harlan) islets were isolated using a rodent-islet isolation technique (59). All animal protocols were approved by the University of Miami Care and Use Committee. Human pancreatic islets were obtained from the Human Islet Cell Processing Facility at the Diabetes Research Institute, University of Miami Miller School of Medicine or from the Islet Cell Resource basic science islet distribution program, Islet Cell Resource Centers (ICRs) Consortium, Division of Clinical Research, National Center for Research Resources, National Institutes of Health. Human islets were dissociated into single cells using enzyme-free cell dissociation buffer (Invitrogen). Islets and islets cells from Q:1 all species were cultured identically (37° C. and 5% CO₂) in CMRL Q:2 medium-1066 (Invitrogen), niacinamide (10 mM; Sigma), ITS (BD Biosciences), Zn₂SO₄ (15 μM, Sigma), GlutaMAX (2 mM; Invitrogen), Hepes (25 mM; Sigma), FBS (10%; Invitrogen), and penicillin-streptomycin (100 IU/mL—100 μg/mL; Invitrogen).

[Ca²⁺]ᵢ Imaging. [Ca²⁺]ᵢ imaging was performed as previously described (8, 36). Dispersed islet cells were immersed in Hepes-buffered solution (125 mM NaCl, 5.9 mM KCl, 2.56 mM CaCl₂, 1 mM MgCl₂, 25 mM Hepes, and 0.1 % BSA, pH 7.4). Glucose was added to give a final concentration of 3 mM. Islets or dispersed islet cells were incubated in Fura-2 AM (2 μM; 1 h) and placed in a closed small volume imaging chamber (Warner Instruments). Stimuli were applied with the bathing solution. Islets loaded with Fura-2 were excited alternatively at 340 and 380 nm with a monochromator light source (Cairn Research Optoscan Monochromator; Cairn Research Ltd). Images were acquired with a Hamamatsu camera (Hamamatsu) attached to a Zeiss Axiovert 200 microscope (Carl Zeiss). Changes in the 340/380 fluorescence emission ratio over time were analyzed in individual islets and dispersed cells using Kinetic Imaging AQM Advance software (Kinetic imaging). Peak changes in the fluorescence ratio constituted the response amplitude. Beta cells were distinguished from other endocrine cells by their [Ca²⁺]ᵢ responses to high glucose concentrations, and alpha cells were identified by their [Ca²⁺]ᵢ responses to kainate (glutamate receptor agonist) (8, 36).

Insulin and Glucagon Secretion. Insulin and glucagon secretion were measured as previously described (8, 36). A high-capacity automated perifusion system was developed to dynamically measure hormone secretion from pancreatic islets. A low pulsatility peristaltic pump pushed Hepes-buffered solution (125 mM NaCl, 5.9 mM KCl, 2.56 mM CaCl₂, 1 mM MgCl₂, 25 mM Hepes, and 0.1% BSA, pH 7.4 at a perifusion rate of 100 μL/min) through a column containing 100 pancreatic islets immobilized in Bio-Gel P-4 Gel (BioRad). Except when otherwise stated, glucose concentration was adjusted to 3 mM for all experiments. Stimuli were applied with the perifusion buffer. The perifusate was collected in an automatic fraction collector designed for a 96-well plate format. The columns containing the islets and the perifusion solutions were kept at 37° C., and the perifusate in the collecting plate was kept at <4° C. Perifusates were collected every 1 min. Hormone release in the perifusate was determined with the human or mouse Endocrine. LINCOplex Kit following manufacturer's instructions (Lincoresearch). Human islet preparations varied considerably in their quality. Thus, the magnitudes of the responses to different stimuli were compared with the same recording or using. recordings from the same preparation.

Immunohistochemistry. Sections (14 μm) were incubated overnight with anti-P2X receptor antibodies (1-7; Alomone Labs), anti-insulin antibodies (1:500; Accurate Chemical & Scientific), antiglucagon antibodies (1:4,000; Sigma), and/or antisomatostatin antibodies (1:1,000, Accurate Chemical & Scientific). As a negative control, purified peptide (50 μg) was preincubated with purinergic receptor primary antibodies (1 μg) for 1 h (room temperature). Pancreatic sections containing islets were examined using a Zeiss LSM 510 scanning confocal microscope (viewed at magnifications ×20 and ×40).

In Situ Hybridization. In situ hybridization using DIG-labeled RNAQ:3 probes for mRNA detection of human P2XRs (1-7) was performed as described (60). A total of 30 ng of DIG-labeled probe was diluted in 150 μl of hybridization buffer, applied to the slides, and allowed to hybridize at 70° C. overnight. Slides were then washed for 1 h at 70° C. in 0.2 SSC solution (Ambion-Q:4 Applied Biosystems) and incubated with alkaline phosphatase-conjugated sheep anti-DIG antibody (Roche) overnight at 4° C. Alkaline phosphatase reaction was carried out in PVA with 200Q:5 μl of MgCl₂ 1 M and 140 μl of NBT/BCIP stock (Roche). SenseQ:6 strand probes were used as a negative control for each P2XR. Immunofluorescence localization of antigens, double-labeled immunofluorescence, and confocal microscopy were carried out as previously described (60). Antibodies used were mouse antiinsulin (1/1,000; Sigma), guinea pig antiglueagon (1/50; Dako), Alexa Fluor 488-conjugated goat anti-mouse (1/400; Molecular Probes), and Alexa Fluor 568-conjugated goat anti-guinea pig (1/400; Molecular Probes). DAPI was used as nuclear counterstaining. Hybridization and immunofluorescence signals were merged by digitally converting the chromogen signal into a color signal in RGB scale. The hybridization signal was pseudocolored in red.Q:7 This signal was then merged with the insulin signal (green). Both transformations were done using Photoshop.

Western Blotting. Immunoblot analysis was carried out by standard methods using the antibodies used for P2X immunohistochemistry (1:1,000). In control experiments, primary antibodies were incubated with corresponding control peptide (Alomone Labs) at a ratio of 50 μg antigenic peptide/1 μg antibody at room temperature for 5 h.

Statistical Analyses. For statistical comparisons, we used a Student t test or a one-Way ANOVA followed by multiple comparison procedures with the Bonferroni t test. Throughout the application, data are presented as average±SEM.

### EXAMPLE 1

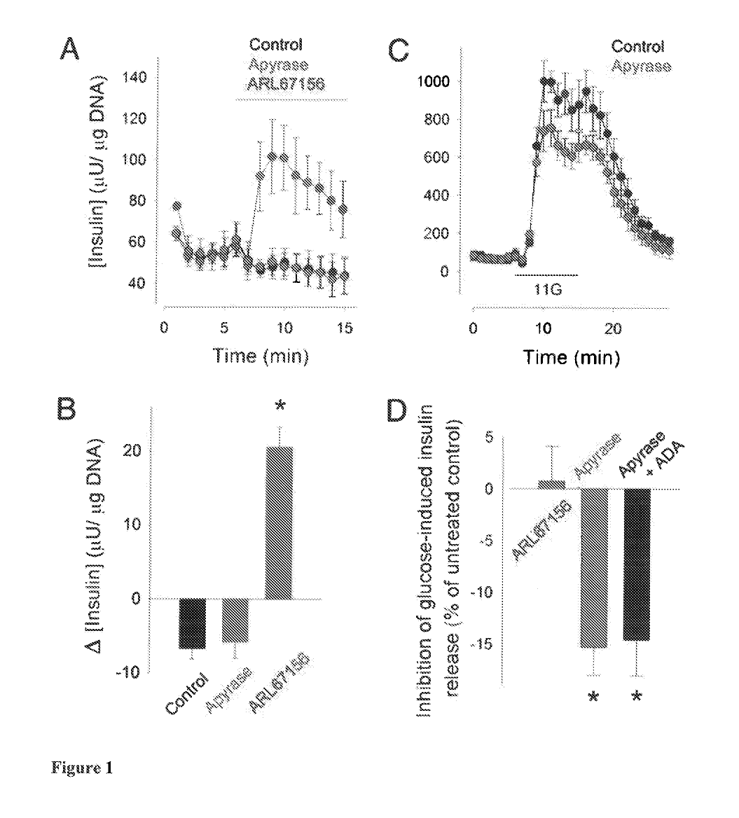

To infer the role of ATP as an autocrine/paracrine signal, we manipulated ATP degradation and thus, the concentration of endogenously released ATP in isolated human islets and recorded changes in hormone secretion by using a perifusion assay of dynamic secretory responses (36). Released ATP is rapidly cleared by membrane ecto-ATPase, such as apyrase, that converts ATP into adenosine (37, 38). Ecto-ATPases are crucial in the duration and magnitude of purinergic signaling (39). A functional apyrase (CD39) has been shown to be expressed in human β cells (40). Application of the apyrase inhibitor ARL67156 (50 μM) (41, 42) increased basal insulin secretion from islets incubated at low glucose concentration (3 mM; FIG. 1 A and B), revealing that human islet cells released ATP. Under these conditions, the endogenous ecto-ATPases are fully effective, explaining why exogenously added apyrase (5 U/mL) did not reduce basal insulin secretion (FIGS. 1 A and B).

### EXAMPLE 2

Because ATP is already released at low glucose concentrations and has the potential to evoke insulin secretion, we hypothesized that ATP potentiates glucose-induced insulin secretion at early stages. Accordingly, exogenously added apyrase (5 U/mL), during a step increase in glucose concentration from 3 mM to 11 mM, reduced insulin release by ˜15% (FIGS. 1 C and D), indicating that endogenously released ATP contributed to the β-cell response. Adding the competitive apyrase inhibitor ARL67156 during glucose stimulation, however, did not amplify the β-cell response (FIG. 1D), suggesting that endogenously released ATP was high enough to saturate its potentiating effect. Hence, stimulating with exogenous ATP while the glucose concentration was increased did not add to the insulin response.

Apyrase may decrease glucose-induced insulin release either by reducing extracellular ATP or by increasing adenosine this may act on P1 receptors to inhibit insulin release (43). Degrading adenosine with adenosine deaminase did not change the effect of apyrase on glucose-stimulated insulin secretion (FIG. ID), indicating that the presence of adenosine did not contribute to the inhibition of the insulin response. Accordingly, neither the P1 receptor antagonist CGS15943 (10 μM) nor adenosine (100 μM) altered glucose-induced insulin secretion (Discussion). Because nerves are severed and neuronal remnants that could be additional sources or targets for ATP do not survive under our experimental conditions (32, 44), the most likely interpretation is that ATP secreted by β cells provides a positive autocrine feedback loop to amplify insulin secretion.

### EXAMPLE 3

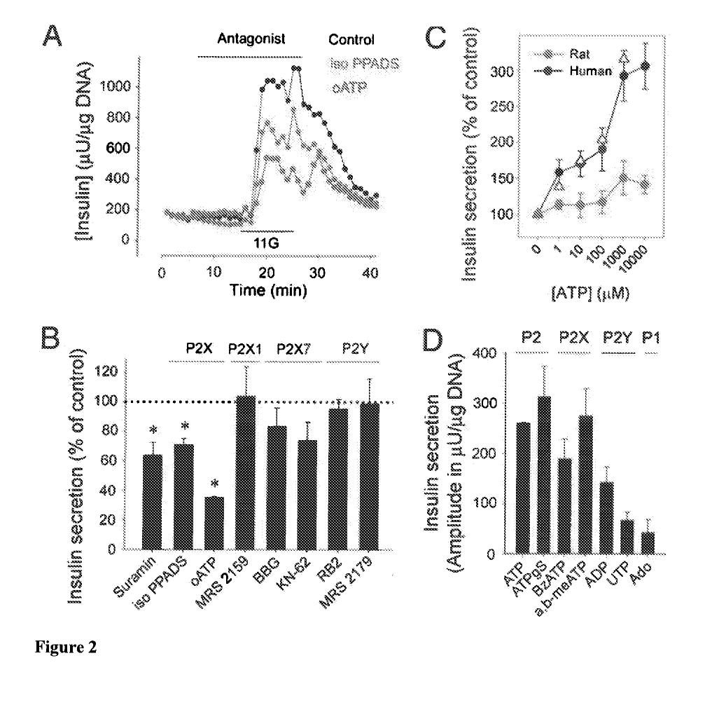

To examine the receptors involved in this autocrine feedback loop, we blocked purinergic receptors with specific receptor antagonists during stimulation with an increase in glucose concentration from 3 mM to 11 mM (FIG. 2A). Insulin secretory responses to glucose stimulation were reduced in the presence of suramin (50 μM; a broad antagonist of P2 receptors), iso-PPADSQ:9 (50 μM; an antagonist for P2X1, P2X2, P2X3, and P2X5 receptors), and oxidized ATP (oATP; 500 μM; an antagonist for P2X2, P2X3, and P2X7 receptors) by 40%, 30%, and 65%, respectively (FIG. 2B). Insulin secretory responses to glucose stimulation in the presence of the specific P2X1 antagonist MRS2159 (10 μM) and the two P2X7 receptor antagonists brilliant Blue G (1 μM) and KN-62 (1 μM) were not significantly reduced (FIG. 2B). Antagonists for P2Y receptors [reactive blue 2 (50 μM) and MRS2179 (10 μM); specific for the P2Y1 receptor; FIG. 2B)] or the P1 receptor [CGS15943 (10 μM)] did not inhibit glucose induced insulin release.

### EXAMPLE 4

To determine the direct effects of purinergic receptor activation on insulin secretion, we applied exogenous ATP and other agonists. In human islets, application of ATP, the universal agonist of P2 purinergic receptors, stimulated increases in insulin release concentration dependently at low (3 mM) and high glucose concentrations (11 mM) with similar thresholds (FIG. 2C). The concentration-response relationship showed a high affinity component (˜0.5 μM) that compared well with the reported EC50 for the human P2X3 receptor (˜0.39 μM) and a second increase between 100 and 1000 μM that might correspond to activation of P2X7 receptors (EC₅₀˜100 μM) (45). Increasing extracellular ATP>1 mM did not further raise insulin release (FIG. 2C). The insulin responses to ATP showed similar increases above basal as the responses stimulated by glucose. Compared with the increase elicited by ATP (1 mM), the response to high glucose (11 mM) was 101%±30% or almost identical. Similar results were obtained using monkey islets. By contrast, neither ATP nor any of the other purinergic agonists tested stimulated insulin release in pig, mouse, or rat islets (FIG. 2C and FIG. S2). In rat islets, only high concentrations of ATP (1 mM) induced small increases in insulin release (FIG. 2C).

ATPγS (50 μM; a nonhydrolysable ATP analog), the specific P2X receptor agonist BzATP (50 μM), and the P2X₁ and P2X₃ agonist α,β-methylene ATP (50 μM) elicited strong insulin responses (FIG. 2D). P2Y receptors were not involved in the response to endogenously released ATP during glucose stimulation but could be directly activated by the selective agonists UTP (100 μM; an agonist of P2Y₂, P2Y₄, and P2Y₆) and ADP (100 μM; an agonist of P2Y₁, P2Y₁₂, and P2Y₁₃) to increase insulin release (FIG. 2D), suggesting the presence of multiple ATP receptor subtypes in the human β cell. Adenosine had a minor effect on insulin release, indicating that P1 receptors were only modestly involved (FIG. 2D). The magnitudes of the insulin responses to ATP (100 μM), ATPγS (50 μM), BzATP (50 μM), UTP (100 μM), and ADP (100 μM) in islets kept at high glucose (11 mM) were similar to the magnitudes of insulin responses to these agonists that were recorded in islets kept at low glucose concentrations, indicating that the effects of purinergic receptor activation are not altered at higher glucose levels.

### EXAMPLE 5

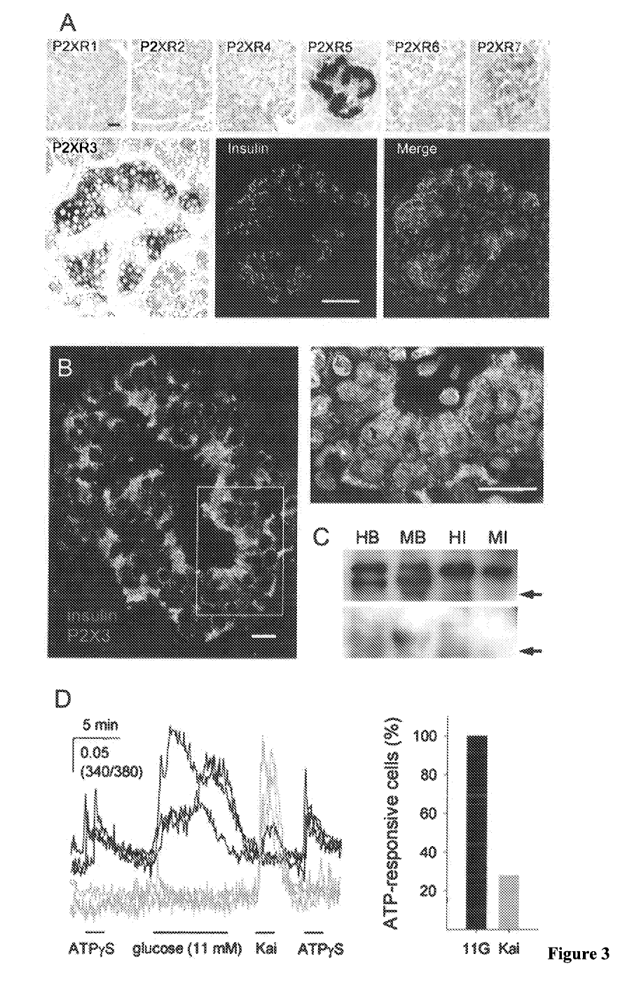

Our results suggest that human islets express P2X receptors with activation that strongly stimulates insulin secretion. By using RTPCR, we found that all P2X receptor genes were expressed in human islets, confirming results from the Beta Cell Biology Consortium database (website:betacell.org/resources/data/epcondb/). To localize P2X receptor expression in the islet, we performed in situ hybridization on human pancreatic sections. Strong hybridization signals in human islets were detected for P2X₃, P2X₅, and P2X₇ (FIG. 3A). By combining in situ hybridization with immunofluorescence for islet hormones, we found that these receptors were expressed in β cells (FIG. 3A). No signals could be detected with P2X₁, P2X₂, P2X4, P2X₆, or control sense riboprobes. Immunofluorescence and Western blots further showed that the P2X3 protein was present in β cells (FIGS. 3B and C). Although P2X5 and P2X7 immunoreactivities were seen in islets, they could not be blocked by control peptide preadsorption. Therefore, it was not possible to determine if the staining could be considered a reliable indication of P2X5 and P2X7 receptor protein expression. Isolated human islet cells were examined for the presence of functional P2X receptors using measurements of [Ca²⁺]ᵢ. Beta cells, identified by their response to high glucose (11 or 16 mM) (8), responded to ATPγS (50 μM) and BzATP (50 μM) with rapid [Ca²⁺]ᵢ increases (FIG. 3D). A fraction of the cells (30%) that responded to the alpha cell-specific stimulus kainate (100 μM) (8) responded to ATPγS (50 μM) or BzATP (50 μM) with rapid [Ca²⁺]ᵢ increases (FIG. 3D). In line with these results, ATP stimulated small increases in glucagon secretion in human, monkey, and mouse islets, and a subset of human alpha cells expressed P2X4 receptors (FIG. S3).

### EXAMPLE 6

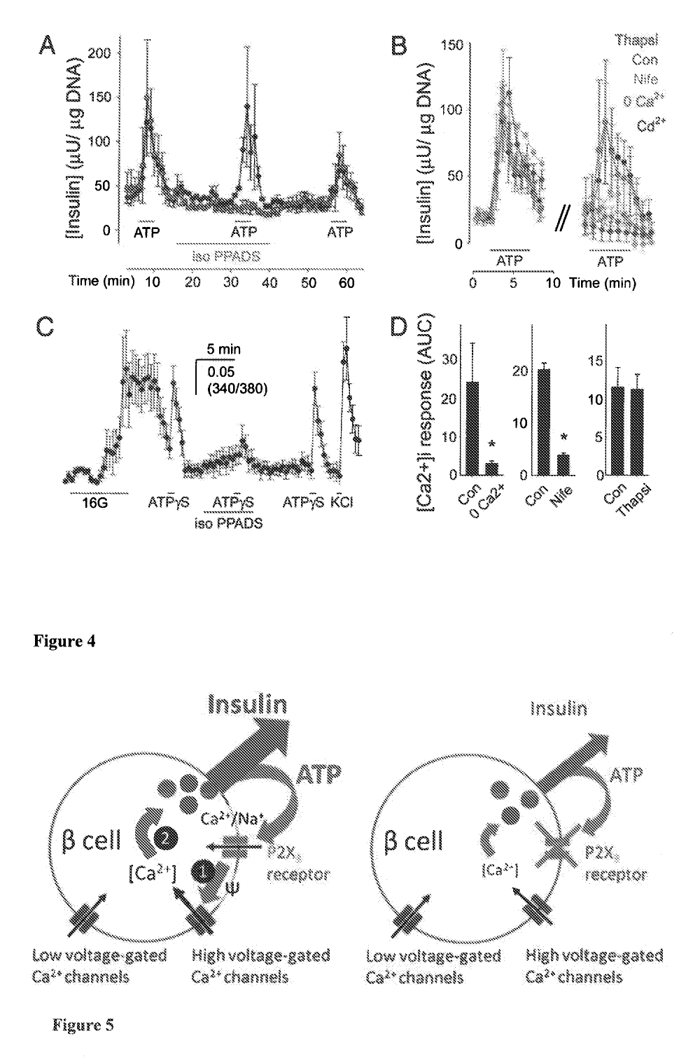

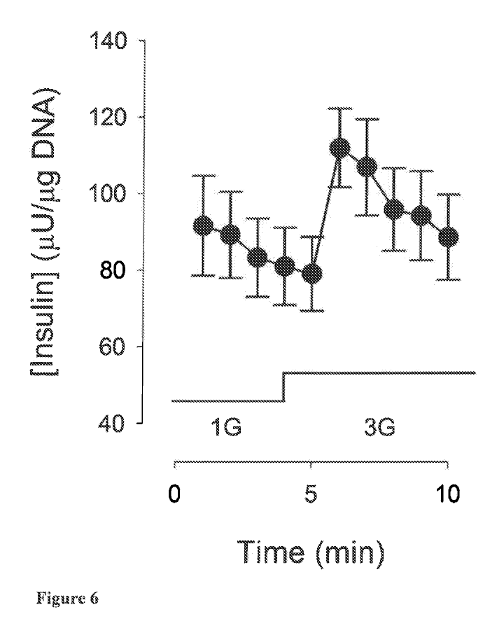

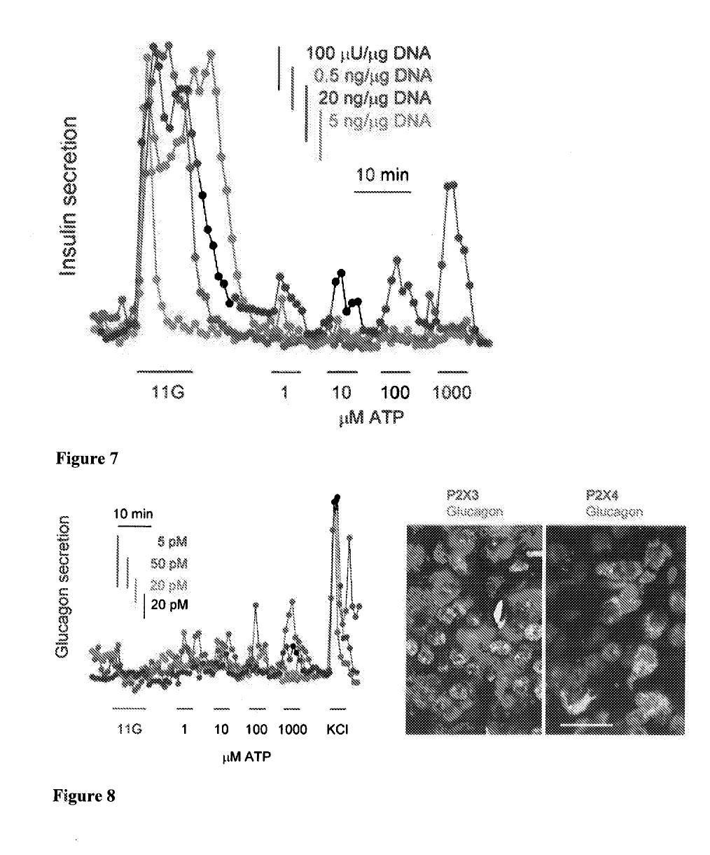

What are the mechanisms by which ATP induces insulin secretion in human β cells? Insulin responses to ATP (10 μM) were inhibited by the general P2 receptor antagonist suramin (100 μM) and the specific P2X antagonist iso-PPADS (50 μM; ˜95% inhibition; FIG. 4A). In the nominal absence of extracellular Ca2+, insulin responses to ATP (FIG. 4B) and α,β meATP (100 μM) were strongly diminished. By contrast, blocking the contribution of Ca²⁺ release from intracellular stores with thapsigargin (1 μM) had no effect on insulin responses to ATP (FIG. 4B). The Ca²⁺ needed for ATP-induced insulin secretion could enter through the P2X receptor pore or voltage-dependent Ca²⁺ channels, which are activated as a consequence of P2X receptor-mediated membrane depolarization. The broad-spectrum voltage-gated Ca2+ channel blocker Cd²⁺ (100 μM; a concentration not affecting Ca²⁺ influx through P2X receptors) (46, 47) and the L-type Ca²⁺ channel blocker nifedipine (10 μM) abolished insulin responses to ATP (FIG. 4B) or α,β meATP.

### EXAMPLE 7

That ATP failed to increase insulin secretion in the presence of Cd²⁺ or nifedipine indicates that P2X receptor activation caused sufficient depolarization to activate voltage-dependent Ca²⁺ channels (15, 17, 47), particularly L-type Ca²⁺ channels critical to the potential firing in human βcells (48). ATP and the P2X receptor agonists BzATP and α,β meATP elicited repeatable [Ca₂₊]ᵢ responses in β cells that were comparable with responses to glucose or KCl stimulation (FIG. 4C). [Ca²⁺]ᵢ responses to ATP were blocked by isoPPADS by ˜80% in human β cell's (FIG. 4C). Thapsigargin (1 μM) did not affect [Ca²⁺]ᵢ responses to ATP, indicating little contribution of Ca²⁺ released from intracellular stores (FIG. 4D). The nominal absence of extracellular Ca²⁺ or the addition of nifedipine (10 μM) reduced [Ca²⁺]ᵢ responses to ATP FIG. 4D), indicating a major Ca²⁺ influx through the β-cell plasma membrane.

### Discussion

The results detailed above demonstrate that human β cells express receptors for extracellular ATP to mediate an essential positive autocrine feedback loop for insulin secretion. We have presented evidence that this autocrine feedback loop is present in human and nonhuman primate islets but not in the other species that we examined. These results support the conclusion that, in primates, P2X receptors predominate in the ATP (purinergic) signaling pathways, amplifying the secretion of insulin in response to rapid increases in glucose concentration (FIG. 5).

Our findings have revealed a signaling pathway for ATP in human β cells. We have found that ATP is already released at low glucose concentrations, which is in agreement with recent studies in rodents showing that ATP can be released from secretory granules while insulin is retained (12, 34). Therefore, ATP signaling may precede secretion of insulin, sensitizing the ₁₃ cell to respond appropriately to glucose stimulation. This notion is in line with studies showing that ATP facilitates neurotransmitter release in presynaptic nerve terminals (49, 50). Our results further suggest that ATP release seems to be strongest during sharp increases in glucose concentration. Although exogenous ATP promoted strong responses in islets kept at constant glucose concentrations (3 mM or 11 mM), it was not effective during abrupt increases in glucose concentration, indicating that the receptors were fully activated by endogenously released ATP under these conditions.

Thus, we have demonstrated that ATP is a signal serving in an autocrine positive feedback loop for insulin release subsequent to glucose stimulation. Our results showing substantial differences between human β cells and rodent β cells in terms of ATP signaling reiterate that the structure and function of the human islets are distinctive (31, 32). Our studies revealed that ATP is a potent stimulator of insulin release in islets of primate species but not in those of the other examined species. Because we used the same technical approach for all species tested, the most likely explanation is that ATP signaling differs between species.

The differences in purinergic signaling suggest that β cells of various species express different subsets of purinergic receptors. Our results show that both P2X and P2Y receptors can be activated in human β cells, but the responses mediated by P2X receptors predominate. In mice, ATP elicits [Ca2+]i responses in β cells predominantly through P2Y receptors, not P2X receptors (26, 51). There are only a few studies examining the expression of P2X receptors in the endocrine pancreas of any species. Recently, P2X1 and P2X3 receptors were identified in isolated single mouse β cells (30), and P2X1, P2X2, P2X3, P2X4, and P2X6 have been detected in the mouse and rat pancreas (28, 52, 53).

Without being bound to any theory of the mechanism of the invention, P2X3 receptors most likely contribute to shape the electric activity of human β cells. Direct application of ATP at 3 mM glucose elicited large insulin and [Ca²⁺]ᵢ responses that were comparable with those elicited by high glucose or KCl depolarization. Blocking ATP receptors with P2X receptor antagonists reduced the insulin response to high glucose by up to 65% (FIG. 2), revealing a strong contribution of ATP receptor activation to the response.

Our results further indicated that most of the human β-cell response to ATP was mediated by ionotropic P2X receptors (FIG. 4). This activation promotes considerably large inward currents in the nA range and thereby, depolarizes the β-cell membrane, which results in increased electric activity (30). However, the exact magnitude of the currents will depend on the amount of ATP released, the receptor density, and/or their localization. By using a combination of technical approaches, we have consistently identified P2X3 receptors in human β cells. P2X1, P2X2, P2X4, and P2X6 receptors, reported to be expressed in rodent β cells (28, 30, 52, 53), could not be detected in human β cells. In contrast, our studies revealed the presence of P2X5 and P2X7. Therefore, P2X receptors in human β cells may exist as monomers or heteromers of combinations of P2X3, P2X5, and P2X7. The presence of a polymorphism at a critical position in the human P2X5 gene indicates that only a small subset of humans (˜14%) will process and translate a functional protein (54, 55), ruling out a contribution of P2X5 to ATP signaling in β cells in most human beings. P2X7 receptors are unlikely to form heteromeric receptors with P2X3 (17) but may work as homomeric receptors. Homomeric P2X7 receptors, however, likely do not participate in normal β-cell physiology, because their activation requires ATP concentrations>100 μM (17). This is in agreement with our results showing that P2X7 receptor antagonists did not affect the positive autocrine feedback loop mediated by ATP. Under physiological conditions, the most likely scenario is that P2X3 homomeric receptors are mediating the positive autocrine feedback loop for the insulin release that we are describing.

Autocrine loops with positive feedback allow cells to modulate the amplitude and the duration of the signaling response to external stimuli (56). We propose that ATP functions in an automodulatory system that, when activated by an increase in blood glucose, adds speed and sensitivity to the β-cell secretory response.

The β cell secretes ATP along with insulin when the glucose concentration increases. Released ATP then activates P2X3 receptors in the β-cell plasma membrane. Activation of P2X3 receptors leads to membrane depolarization mediated by Ca2+ and Na+ influx (17) and subsequent opening of voltage-gated Ca2+ channels. This results in increased [Ca2+]i and enhanced insulin secretion. This positive feedback allows the β cell to translate small changes in plasma glucose into large alterations in insulin release. Thus, positive ATP autocrine signaling may explain how adequate and fast insulin release can be achieved in response to modest physiological changes in blood glucose concentration.

### REFERENCES

1. Conn P M, Goodman H M, Kostyo J L (1998) The Endocrine System (Oxford University Press, New York), pp 1-5.
2. Doyle M E, Egan J M (2003) Pharmacological agents that directly modulate insulin secretion. Pharmacol Rev 55:105-131.
3. Franklin I K, Wollheim C B (2004) GABA in the endocrine pancreas: Its putative role as an islet cell paracrine-signaling molecule. J Gen Physiol 123:185-190.
4. Ishihara H, Maechler P, Gjinovci A, Herrera P L, Wollheim C B (2003) Islet β-cell secretion determines glucagon release from neighbouring alpha-cells. Nat Cell Biol 5:330-335.
5. Kisanuki K, et al. (1995) Expression of insulin receptor on clonal pancreatic alpha cells and its possible role for insulin-stimulated negative regulation of glucagon secretion. Diabetologia 38:422-429.
6. Leibiger I B, Leibiger B, Berggren P O (2002) Insulin feedback action on pancreatic β-cell function. FEBS Lett 532:1-6.
7. Rorsman P, et al. (1989) Glucose-inhibition of glucagon secretion involves activation of GABAA-receptor chloride channels. Nature 341:233-236.
8. Cabrera O, et al. (2008) Glutamate is a positive autocrine signal for glucagon release. Cell Metab 7:545-554.
9. Detimary P, Jonas J C, Henquin J C (1996) Stable and diffusible pools of nucleotides in pancreatic islet cells. Endocrinology 137:4671-4676.
10. Hazama, A, Hayashi S, Okada Y (1998) Cell surface measurements of ATP release from single pancreatic β cells using a novel biosensor technique. Pflugers Arch 437:31-35.
11. Leitner J W, Sussman K E, Vatter A E, Schneider F H (1975) Adenine nucleotides in the secretory granule fraction of rat islets. Endocrinology 96:662-677.
12. MacDonald P E, Braun M, Galvanovskis J, Rorsman P (2006) Release of small transmitters through kiss-and-run fusion pores in rat pancreatic β cells. Cell Metab 4: 283-290.
13. Burnstock G (2006) Pathophysiology and therapeutic potential of purinergic signaling. Pharmacol Rev 58:58-86.
14. Fields R D, Burnstock G (2006) Purinergic signaling in neuron-glia interactions. Nat Rev Neurosei 7:423-436.
15. Khakh B S, North R A (2006) P2X receptors as cell-surface ATP sensors in health and disease. Nature 442:527-532.
16. Ralevic V, Burnstock G (1998) Receptors for purines and pyrimidines. Pharmacol Rev 50:413-492.
17. North R A (2002) Molecular physiology of P2X receptors. Physiol Rev 82:1013-1067.
18. Edwards F A, Gibb A J, Colquhoun D (1992) ATP receptor-mediated synaptic currents in the central nervous system. Nature 359:144-147.
19. Knott T K, Velázquez-Marrero C, Lemos J R (2005) ATP elicits inward currents in isolated vasopressinergic neurohypophysial terminals via P2X2 and P2X3 receptors. Pflugers Arch 450:381-389.
20. Tomié M, Jobin R M, Vegara L A, Stojilkovic S S (1996) Expression of purinergic receptor channels and their role in calcium signaling and hormone release in pituitary gonadotrophs. Integration of P2 channels in plasma membrane- and endoplasmic reticulum-derived calcium oscillations. J Biol Chem 271:21200-21208.
21. Gu J G, MacDermott A B (1997) Activation of ATP P2X receptors elicits glutamate release from sensory neuron synapses. Nature 389:749-753.
22. Petit P, Manteghetti M, Puech R, Loubatieres-Mariani M M (1987) ATP and phosphate-modified adenine nucleotide analogues. Effects on insulin secretion and calcium uptake. Biochem Pharmacol 36:377-380.
23. Salehi A, Qader S S, Quader S S, Grapengiesser E, Hellman B (2005) Inhibition of purinoceptors amplifies glucose-stimulated insulin release with removal of its pulsatility. Diabetes 54:2126-2131.
24. Léon C, et al. (2005) The P2Y(1) receptor is involved in the maintenance of glucose homeostasis and in insulin secretion in mice. Purinergic Signal 1:145-151.
25. Petit P, et al., (1998) Evidence for two different types of P2 receptors stimulating insulin secretion from pancreatic B cell. Br J Pharmacol 125:1368-1374.
26. Poulsen C R, et al. (1999) Multiple sites of purinergic control of insulin secretion in mouse pancreatic β-ceIls. Diabetes 48:2171-2181.
27. Bertrand G, Chapal J, Loubatières-Mariani M M, Roye M (1987) Evidence for two different P2-purinoceptors on β cell and pancreatic vascular bed. Br J Pharmacol 91:783-787.
28. Richards-Williams C, Contreras J L, Berecek K H, Schwiebert E M (2008) Extracellular ATP and zinc are co-secreted with insulin and activate multiple P2X purinergic receptor channels expressed by islet β-cells to potentiate insulin secretion. Purinergic Signal 4:393-405.
29. Fernandez-Alvarez J. Hillaire-Buys D, Loubatières-Mariani M M, Gomis R, Petit P (2001) P2 receptor agonists stimulate insulin release from human pancreatic islets. Pancreas 22:69-71.
30. Silva A M, et al, (2008) Electrophysiological and immunocytochemical evidence for P2X purinergic receptors in pancreatic β cells. Pancreas 36:279-283.
31. Brissova M, et al, (2005) Assessment of human pancreatic islet architecture and composition by laser scanning confocal microscopy. J Histochem Cytochem 53: 1087-1097.
32. Cabrera O, et al. (2006) The unique cytoarchitecture of human pancreatic islets has implications for islet cell function. Proc Natl Acad Sci USA 103:2334-2339.
33. Braun M, et al. (2007) Corelease and differential exit via the fusion pore of GABA, serotonin, and ATP from LDCV in rat pancreatic β cells. J Gen Plysiol 129:221-231.
34. Obermüller S, et al. (2005) Selective nucleotide-release from dense-core granules in insulin-secreting cells. J Cell Sci 118:4271 1282.
35. Henquin J C, Dufrane D, Nenquin M (2006) Nutrient control of insulin secretion in isolated normal human islets. Diabetes 55:3470-3477.
36. Cabrera O, et al. (2008) Automated, high-throughput assays for evaluation of human pancreatic islet function. Cell Transplant 16:1039-1048.
17. Zimmermann H (2000) Extracellular metabolism of ATP and other nucleotides. Naunyn Schmiedebergs Arch Pharmacol 362:299-309.
38. Cunha R A (2001) Regulation of the ecto-nucleotidase pathway in rat hippocampal nerve terminals. Neurochem Res 26:979-991.
39. Bours M J, Swennen E L, Di Virgilio F, Cronstein B N, Dagnelie P C (2006) Adenosine 5′-triphosphate and adenosine as endogenous signaling molecules in immunity and inflammation. Pharmacol Ther 112:358-404.
40. Kittel A, Garrido M, Varga G (2002) Localization of NTPDase1/CD39 in normal and transformed human pancreas. J Histochem Cytochem 50:549-556.
41. Crack B E, et al. (1995) Pharmacological and biochemical analysis of FPL 67156, a novel, selective inhibitor of ecto-ATPase. Br J Pharmacol 114:475-481.
42. Westfall T D, Kennedy C, Sneddon. P (1997) The ecto-ATPase inhibitor ARL 67156 enhances parasympathetic neurotransmission in the guinea-pig urinary bladder. Eur J Pharmacol 329:169-173.
43. Hillaire-Buys D. Gross R, Paŕes-Herbuté N, Ribes G, Loubatières-Mariani M M (1994) In vivo and in vitro effects of adenosine-5′-O-(2-thiodiphosphate) on pancreatic hormones in dogs. Pancreas 9:646-651.
44. Karlsson S, Myrsén U, Nieuwenhuizen A, Sundler F, Ahrén B (1997) Presynaptic sympathetic mechanism in the insulinostatic effect of epinephrine in mouse pancreatic islets. Am J Physiol 272:R1371-R1378.
45. Bianchi B R, et al, (1999) Pharmacological characterization of recombinant human and rat P2X receptor subtypes. Eur J. Pharmacol 376:127-138.
46. Inoue. K, Koizumi S, Nakazawa K (1995) Glutamate-evoked release of adenosine 5′-triphosphate causing an increase in intracellular calcium in hippocampal neurons. Neuroreport 6:437-440.
47. Khakh B S, Henderson G (1998) ATP receptor-mediated enhancement of fast excitatory neurotransmitter release in the brain. Mol Pharmacol 54:372-378.
48. Braun M, et al. (2008) Voltage-gated ion channels in human pancreatic βcells: Electrophysiological characterization and role in insulin secretion. Diabetes 57:1618-1628.
49. Cunha R A, Ribeiro J A (2000) ATP as a presynaptic modulator. Life Sci 68:119-137.
50. Dorostkar M M, Boehm S (2008) Presynaptic lonotropic receptors. Handb Exp Pharmacol 184:479-527.
51. Hellman B, Dansk H, Grapengiesser E (2004) Pancreatic β-cells communicate via intermittent release of ATP. Am J Physiol Endocrinol Metab 286:E759-E765.
52. Coutinho-Silva R, Parsons M, Robson T, Burnstock G (2001) Changes in expression of P2 receptors in rat and mouse pancreas during development and ageing. Cell Tissue Res 306:373-383.
53. Coutinho-Silva R, Parsons M, Robson T, Lincoln J, Burnstock G (2003) P2X and P2Y purinoceptor expression in pancreas from streptozotocin-diabetic rats. Mol Cell Endocrinol 204:141-154.
54. Lê K T, Paquet M, Nouel D, Babinski K, Séguéla P (1997) Primary structure and expression of a naturally truncated human P2X ATP receptor subunit from brain and immune system. FEBS Lett 418:195-199.
55. Bo X, et al, (2003) Pharmacological and biophysical properties of the human P2X5 receptor. Mol Pharmacol 63:1407-1416.
56. Shvartsman S Y, et al. (2002) Autocrine loops with positive feedback enable context-dependent cell signaling. Am J Physiol Cell Physiol 282:C545-C559,
57. Ricordi C, Lacy P E, Fink E H, Olack B J, Scharp D W (1988) Automated method for isolation of human pancreatic islets. Diabetes 37:413-420.
58. Kenyon N S, et al, (1999) Long-term survival and function of intrahepatic islet allografts in rhesus monkeys treated with humanized anti-CD154. Proc Natl Acad Sci USA 96:8132-8137.
59. Berney T, et al, (2001) Endotoxin-mediated delayed islet graft function is associated with increased intra-islet cytokine production and islet cell apoptosis. Transplantation 71:125-132.
60. Apelqvist A, Ahlgren U, Edlund H (1997) Sonic hedgehog directs specialised mesoderm differentiation in the intestine and pancreas. Curr Biol 7:801-804.

1. A method of increasing insulin secretion in a subject in need thereof, said method comprising administering an effective amount of a P2X purinergic agonist to said subject

2. The method of claim 1 wherein said subject is human.

3. The method of claim 1 wherein said subject is suffering from diabetes mellitus.

4. The method of claim 3 wherein the diabetes mellitus is type 2 diabetes.

5. The method of claim 1 wherein the P2X purinergic agonist is a P2X₃ agonist.

6. The method of claim 1 wherein the P2X purinergic agonist is selected from the group consisting of 2-methylthio-ATP (2-meSATP), 5-bromouridine 5-triphosphate, 3′-O-(4-benzoylbenzoyl)-ATP, and α,β-methylene ATP.

7. The method of claim 1 wherein the dosage of P2X purinergic agonist administered results in a concentration at the target tissue of between about 10 μM and 1 raM, e.g. between about 10 μM and 100 μM.

8-11. (canceled)

12. A pharmaceutical composition comprising an effective amount of a P2X purinergic agonist to stimulate insulin secretion for treatment of diabetes.

13. The pharmaceutical composition of claim 12 wherein the P2X purinergic agonist is a P2X3 agonist.

14. The pharmaceutical composition of claim 13 wherein the P2X3 agonist is selected from the group consisting of 2-methylthio-ATP (2-meSATP), 5-bromouridine 5-triphosphate, 3′-O-(4-benzoylbenzoyl)-ATP, and α,β-methylene ATP.

15. A method of screening for a compound compound/agent effective for increasing insulin secretion in a primate, comprising contacting a test compound with a P2X3 receptor and measuring the activity of the receptor, wherein an increase in activity of the receptor is indicative of a candidate compound effective for increasing insulin secretion.

16. The method of claim 15, wherein the P2X3 receptor is on a cell.

17. The method of claim 16, wherein the activity is measured by measuring insulin secretion from said cell.

18. The method of claim 16, wherein the cell is a pancreatic islet cell.

19. The method of claim 15, wherein the primate is a human.
